(19) United States
(12) Patent Application Publication
(10) Pub. No.: US 2025/0107940 A1
(43) Pub. Date: Apr. 3, 2025

(54) COMPRESSION GARMENT

(71) Applicant: HADDENHAM HEALTHCARE LTD, Aylesbury (GB)

(72) Inventor: Tom Wright, Aylesbury (GB)

(73) Assignee: HADDENHAM HEALTHCARE LTD, Aylesbury (GB)

(21) Appl. No.: 18/903,726

(22) Filed: Oct. 1, 2024

(30) Foreign Application Priority Data
Oct. 2, 2023 (GB) 2315082.4

(51) Int. Cl. A61F 13/06

(52) U.S. Cl. CPC A61F 13/067

(57) ABSTRACT

A foot compression garment for treating lymphedema includes a first bandage portion with a sole portion and a pair of opposed side portions. Each side portion includes a first end attached to the sole portion, and a second free end. The free end includes a first attachment feature to releasably engage the second side portion. A heel portion is permanently attached to the sole portion and located between the pair of opposed side portions. A second bandage portion includes a pair of opposed second bandage portion members, each having a first end attached to the heel portion, and a second free end. The second free end includes a second attachment feature for releasable engagement to the first bandage portion. The heel portion includes spaced apart side portions with a central portion therebetween. The second bandage portion members are attached at the corresponding side portions of the heel member.

### CROSS-REFERENCE TO RELATED APPLICATION

This application which claims the benefit of United Kingdom Patent Application GB 2315082.4, filed on Oct. 2, 2023, the content of which is incorporated in its entirety.

### TECHNICAL FIELD

The present disclosure relates to a compression garment for the treatment of lymphedema in the foot of a user. In particular, the present disclosure relates to a single piece compression garment for the treatment of lymphedema in the foot of a user. The present disclosure also relates to a method for producing the single piece compression garment.

### BACKGROUND

Compression garments are used to treat a variety of conditions, most notably swelling and oedema, in which a higher than normal amount of fluid accumulates in the interstitium beneath the skin of a patient.

Conventional compression garments for use with the foot of a user have been found to be difficult to mount and can give rise to pressure sores on the posterior ankle as a result of compressive forces being supplied to this area.

There is therefore a need for an improved compression garment for use on a foot of a user.

### SUMMARY

According to a first aspect there is provided a single piece foot compression garment for the treatment of lymphedema in the foot of a user, the foot compression garment comprising:
a first bandage portion composed of elastic material, in which the first bandage portion comprises:
a sole portion configured in use to be positioned adjacent a sole of a foot of a user;
a pair of opposed side portions comprising a first side portion and a second side portion, each side portion comprising a first end permanently attached to the sole portion and extending laterally therefrom, and a second opposed free end, and in which the second opposed free end of the first side portion comprises at least one first attachment feature configured in use to releasably engage the second side portion; and
a heel portion extending from and permanently attached to the sole portion and located between the pair of opposed side portions, in which the heel portion is configured in use to be positioned adjacent a heel of a foot of a user; and
a second bandage portion composed of elastic material, in which the second bandage portion comprises a pair of opposed second bandage portion members, each second bandage portion member having a first end permanently attached to the heel portion, and a second opposed free end, in which the second opposed free end of at least one second bandage portion member comprises a second attachment feature configured in use for releasable engagement to a corresponding side portion of the first bandage portion,
in which the heel portion comprises a pair of spaced apart side portions with a central portion extending therebetween, and in which the second bandage portion members are permanently attached at or adjacent the corresponding side portions of the heel member and extending laterally therefrom such that the central portion of the heel portion is free from contact or overlap with the second bandage portion members.

The central portion of the heel portion is preferably a single layer of material.

The second bandage portion is preferably configured in use to extend across and to apply compressive forces to the dorsum and anterior ankle of a foot of a user.

The second bandage portion is preferably configured in use to not extend across or apply compressive forces to the posterior ankle of a foot of a user.

The heel portion preferably comprises a lower end permanently attached to the sole portion, an opposed upper end, and opposed side portions extending therebetween. The central portion is preferably located between the side portions and the upper and lower end of the heel portion. Each second bandage portion member is preferably attached to the heel portion from at or adjacent the upper end to at or adjacent the lower end of the heel portion.

In one embodiment, the distance between the first end of a first second bandage portion member and the first end of a second second bandage portion member is at least 2.0 cm, preferably at least 2.5 cm, for example about 3 cm. The distance between the first end of a first second bandage portion member and the first end of a second second bandage portion member is preferably no more than 5.0 cm, preferably no more than 4 cm, for example no more than 3.5 cm.

The central portion is preferably extendable in a direction extending between the lower and upper ends of the heel portion. The central portion may comprise an extendable region, for example an elasticated region.

The elongation of the central portion of the heel portion of the first bandage portion, in a direction extending in a direction between the lower and upper ends thereof, is preferably at least 110%, for example 115%, of the unstretched length of the central portion of the heel member.

The second bandage portion is preferably attached to the heel portion of the first bandage portion by stitching or mechanical bonding.

The first and/or second bandage portion is preferably composed of a textile fabric. For example, the textile fabric preferably comprises polyamide and elastane. In one embodiment, the textile fabric comprises 87% polyamide and 13% elastane based on the weight of the textile fabric.

Preferably, at least one of the first and/or second attachment feature comprises hook and eye fasteners (e.g. Velcro™).

According to a second aspect of the present invention, there is provided a method for producing a single piece foot compression garment comprising:
forming a first bandage portion composed of elastic material, comprising:
obtaining a sole portion configured in use to be positioned adjacent a sole of a foot of a user;
obtaining a pair of opposed side portions comprising a first side portion and a second side portion, each side portion comprising a first end and a second opposed end, in which the second opposed end of the first side portion comprises at least one first attachment feature;
attaching the first end of each of the first and second side portions to a corresponding side portion of the sole portion such that the first and second side portions extend laterally therefrom such that the at least one first attachment feature is configured in use to releasably engage the second side portion; and
obtaining a heel portion attached to and extending from the sole portion and located between the pair of opposed side portions, in which the heel portion is configured in use to be positioned adjacent a heel of a foot of a user; and
forming a second bandage portion composed of elastic material, comprising:
obtaining a pair of opposed second bandage portion members, each second bandage portion member having a first end, and an opposed second end comprising an attachment feature configured in use for releasable engagement to a corresponding side portion of the first bandage portion;
permanently attaching the first end of each second bandage portion member at or adjacent a corresponding side portion of the heel portion of the first bandage portion such that a central portion of the heel portion is free from contact or overlap with the second bandage portion members.

According to a further aspect of the present invention, there is provided a method for applying a single piece foot compression garment as herein described comprising:
positioning the first bandage portion such that the sole portion is positioned adjacent a sole of a foot of a user, and the heel portion is positioned adjacent a heel of a foot of a user;
extending the pair of opposed side portions across the upper portion of the foot of the user to provide a predetermined compressive force to the underlying region of the foot, and engaging the at least one first attachment feature of the second end of the first side portion to the second side portion; and
extending each of the second bandage portion members across the dorsum and anterior ankle of the foot of a user to provide a predetermined compressive force to the underlying region of the foot, and securing the attachment feature of the at least one second bandage portion member to a corresponding side portion of the first bandage portion.

Embodiments of the present invention will now be described in more detail in relation to the accompanying Figures.

### BRIEF DESCRIPTION OF THE DRAWINGS

FIG. 1 is a photograph of a single piece foot compression garment according to one embodiment of the present invention prior to mounting on a foot of a user;

FIG. 2 is a photograph of the single piece foot compression garment of FIG. 1 mounted on a foot of a user; and

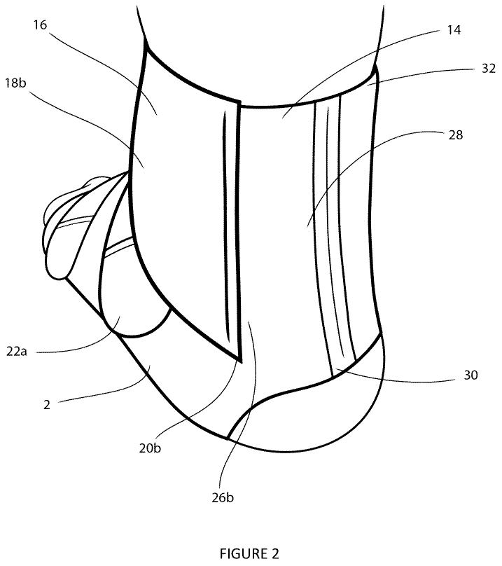

FIG. 3 is a photograph of the central portion of the heel portion of the single piece foot compression garment of FIG. 1 being stretched in a direction extending between the upper and lower ends thereof.

### DETAILED DESCRIPTION

With reference to the Figures, the single piece foot compression garment 1 of the present invention comprises a first bandage portion 2 composed of elastic material. The first bandage portion 2 comprises a sole portion 4 configured in use to be positioned adjacent a sole of a foot of a user. The first bandage portion 2 further comprises a pair of opposed side portions 6a, 6b comprising a first side portion 6a and a second side portion 6b. Each side portion 6a, 6b comprises a first end 8a, 8b permanently attached to the sole portion 4 and extending laterally therefrom, and a second opposed free end 10a, 10b. The second opposed free end 10a of the first side portion 6a comprises a pair of spaced apart first attachment features 12 configured in use to releasably engage the second side portion 6b.

It is to be understood that the side portions 6a, 6b may have any suitable shape. Preferably, the side portions 6a, 6b are elongate in shape. In the illustrated embodiment, each side portion is substantially V-shaped, and each free end of the V-shaped side portion provides a first attachment feature 12.

The first bandage portion 2 further comprises a heel portion 14 extending from and permanently attached to the sole portion 4. The heel portion 14 is located between the pair of opposed side portions 6a, 6b. The heel portion 14 is configured in use to be positioned adjacent a heel of a foot of a user; and

The single piece foot compression garment 1 further comprises a second bandage portion 16 which is composed of elastic material. The second bandage portion 16 comprises a pair of opposed second bandage portion members 18a, 18b. Each second bandage portion member 18a, 18b has a first end 20a, 20b permanently attached to the heel portion 14, and a second opposed free end 22a, 22b. The second opposed free end 22a, 22b of each second bandage portion member 18a, 18b comprises a second attachment feature 24 configured in use for releasable engagement to a corresponding side portion 6a, 6b of the first bandage portion 2.

In the illustrated embodiment, the first attachment feature and the second attachment feature 24 comprise hook and loop fasteners, for example Velcro™. It is however to be understood that the first and second attachment features may be provided by any suitable releasable fastenings.

The heel portion 14 comprises a pair of spaced apart side portions 26a, 26b with a central portion 28 extending therebetween. The second bandage portion members 18a, 18b are permanently attached at or adjacent the corresponding side portions 26a, 26b of the heel member 14 and extend laterally therefrom such that the central portion 28 of the heel portion 14 is free from contact or overlap with the second bandage portion members 18a, 18b.

The heel portion 14 comprises a lower end 30 permanently attached to the sole portion 4, an opposed upper end 32, with the opposed side portions 26a, 26b extending therebetween. The central portion 28 of the heel portion 14 is located between the side portions 26a, 26b and the upper 30 and lower 32 end of the heel portion 14. In the illustrated embodiment, each second bandage portion member 18a, 18b is attached to the heel portion 14 from at or adjacent the upper end 32 to at or adjacent the lower end 30 of the heel portion 14. It is however to be understood that the second bandage portion members 18a, 18b may be attached across a portion of the material extending between the upper 32 and lower 30 ends of the heel portion 14.

The second bandage portion members 18a, 18b are attached to the heel portion 14 by stitching or mechanical bonding.

As a result of the positioning of the second bandage portion members 18a, 18b, the central portion 28 of the heel portion 14 is a single layer of material. The first bandage portion 2 and the second bandage portion members 18a, 18b are composed of a textile material. For example, the textile fabric preferably comprises polyamide and elastane. In one embodiment, the textile fabric comprises 87% polyamide and 13% elastane based on the weight of the textile fabric.

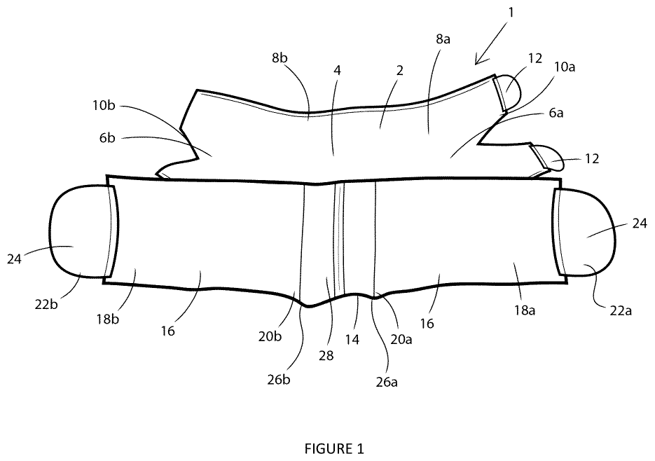

As shown in FIG. 1, the distance between the first end of a first second bandage portion member and the first end of a second second bandage portion member is at least 2.0 cm. It is however to be understood that the spacing may be between 2.0 cm and 5.0 cm.

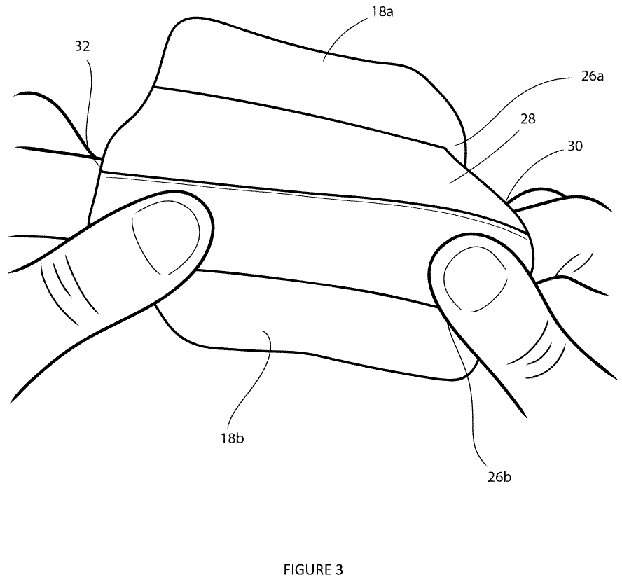

As shown in FIG. 3, the central portion 28 of the heel portion 14 of the first bandage portion 2 can be easily stretched in a direction extending in a direction between the lower 30 and upper 32 ends thereof. Preferably, the central portion 28 of the heel portion 14 is capable of being stretched (in a direction between the lower 30 and upper 32 ends thereof) by an amount at least 110% of the unstretched length of the central portion of the heel portion 14.

In use, a user positions the first bandage portion 2 such that the sole portion 4 is positioned adjacent a sole of a foot of a user, and the heel portion 14 is positioned adjacent a heel of a foot of a user. The user then extends the pair of opposed side portions 6a, 6b across the upper portion of the foot of the user to provide a predetermined compressive force to the underlying region of the foot. The user then releasably engages each of the first attachment features 12 provided by the second end 10a of the first side portion 6a to the second side portion 6b.

The user then extends each of the second bandage portion members 18a, 18b across the dorsum and anterior ankle of the foot of a user to provide a predetermined compressive force to the underlying region of the foot. The second bandage portion members 18a, 18b are then secured in position by releasably engaging the second attachment feature 24 of the second bandage portion member 18a, 18b to a corresponding side portion 6a, 6b of the first bandage portion 2.

The second bandage portion 16 is configured in use to extend across and to apply compressive forces to the dorsum and anterior ankle of a foot of a user. The second bandage portion 16 is configured in use to not extend across or apply compressive forces to the posterior ankle of a foot of a user.

Conventional compression garments for use on a foot of a user are typically rather bulky in size around the posterior ankle. As a result, the conventional compression garments can exert a significant compressive force on the posterior ankle tendon and this can lead to pressure sores and giving rise to an associated risk of infection.

The single piece compression garment of the present invention comprises two spaced apart second bandage portion members 18a, 18b which are permanently attached by for example stitching or mechanical bonding to the first bandage portion 2. By spacing the second bandage portion members 18a, 18b apart laterally, this ensures that the second bandage portion 16 does not extend across the central portion of the heel portion. As a result, the single piece compression garment utilises less material positioned adjacent the posterior ankle tendon than conventional compression garments. As a result, the single piece compression garment of the present invention exerts significantly reduces compressive forces on to the posterior ankle than conventional compression garments. The compression garment of the present invention therefore reduces the risk of pressure sores arising and thereby reduces the risk of an associated infection.

The compression garment of the present invention is easier to mount on a foot of a user as a result of the garment being provided as a single piece. The single piece garment reduces the risk of parts being lost or connected incorrectly during mounting of the garment.

The compression garment of the present invention provides a single layer heel portion whilst also enabling the second bandage portion members to stretch across and be fastened over the dorsum and anterior ankle of a user. As a result of the second bandage portion members not extending across the central portion of the heel portion, the heel portion is extendible in a direction extending between the lower and upper ends thereof. The stretchability of the heel portion therefore allows for improved fitting of the compressive garment to individual feet. This reduces the risk of the garment being poor fitting or providing reduced compressive forces.

Additionally, the presence of a single layer heel portion (without the added bulk of a second bandage portion being located across the heel portion) makes the use of normal footwear easier.

What is claimed is:

1. A single piece foot compression garment for treating lymphedema in a foot of a user, the single piece foot compression garment comprising:
a first bandage portion composed of elastic material, in which the first bandage portion comprises:
a sole portion configured in use to be positioned adjacent a sole of a foot of a user;
a pair of opposed side portions comprising a first side portion and a second side portion, each side portion comprising a first end permanently attached to the sole portion and extending laterally therefrom, and a second opposed free end, and in which the second opposed free end of the first side portion comprises at least one first attachment feature configured in use to releasably engage the second side portion; and
a heel portion extending from and permanently attached to the sole portion and located between the pair of opposed side portions, in which the heel portion is configured in use to be positioned adjacent a heel of a foot of a user; and
a second bandage portion composed of elastic material, in which the second bandage portion comprises a pair of opposed second bandage portion members, each second bandage portion member having a first end permanently attached to the heel portion, and a second opposed free end, in which the second opposed free end of at least one second bandage portion member comprises a second attachment feature configured in use for releasable engagement to a corresponding side portion of the first bandage portion,
in which the heel portion comprises a pair of spaced apart side portions with a central portion extending therebetween, and in which the second bandage portion members are permanently attached at or adjacent the corresponding side portions of the heel portion and extending laterally therefrom such that the central portion of the heel portion is free from contact or overlap with the second bandage portion members.

2. The single piece foot compression garment as claimed in claim 1,
in which the central portion of the heel portion is a single layer of material.

3. The single piece foot compression garment as claimed in of claim 1,
in which the second bandage portion is configured in use to extend across and to apply compressive forces to the dorsum and anterior ankle of a foot of a user.

4. The single piece foot compression garment as claimed in claim 1,
in which the second bandage portion is configured in use to not extend across or apply compressive forces to the posterior ankle of a foot of a user.

5. The single piece foot compression garment as claimed in claim 1,
in which the heel portion comprises a lower end permanently attached to the sole portion, an opposed upper end, and opposed side portions extending therebetween, in which the central portion is located between the side portions and the upper and lower end of the heel portion, and in which each second bandage portion member is attached to the heel portion from at or adjacent the upper end to at or adjacent the lower end of the heel portion.

6. The single piece foot compression garment as claimed in claim 1,
in which a distance between the first end of a first second bandage portion member and the first end of a second second bandage portion member is at least 2.0 cm.

7. The single piece foot compression garment as claimed in claim 1,
in which an elongation of the central portion of the heel member is at least 110% of an unstretched length of the central portion of the heel member.

8. The single piece foot compression garment as claimed in claim 1,
in which the second bandage portion is attached to the heel portion of the first bandage portion by stitching or mechanical bonding.

9. The single piece foot compression garment as claimed in claim 1, in which the first bandage portion and/or the second bandage portion is composed of a textile fabric.

10. The single piece foot compression garment as claimed in claim 9,
in which the textile fabric comprises polyamide and elastane.

11. The single piece foot compression garment as claimed in claim 10,
in which the textile fabric comprises 87% polyamide and 13% elastane based on a weight of the textile fabric.

12. The single piece foot compression garment as claimed in claim 1,
in which at least one of the first attachment feature and/or the second attachment feature comprises hook and eye fasteners (e.g. Velcro™).

13. A method for producing a single piece foot compression garment comprising:
forming a first bandage portion composed of elastic material, comprising:
obtaining a sole portion configured in use to be positioned adjacent a sole of a foot of a user;
obtaining a pair of opposed side portions comprising a first side portion and a second side portion, each side portion comprising a first end and a second opposed end, in which the second opposed end of the first side portion comprises at least one first attachment feature;
attaching the first end of each of the first and second side portions to a corresponding side portion of the sole portion such that the first and second side portions extend laterally therefrom such that the at least one first attachment feature is configured in use to releasably engage the second side portion; and
obtaining a heel portion attached to and extending from the sole portion and located between the pair of opposed side portions, in which the heel portion is configured in use to be positioned adjacent a heel of a foot of a user; and
forming a second bandage portion composed of elastic material, comprising:
obtaining a pair of opposed second bandage portion members, each second bandage portion member having a first end, and an opposed second end comprising an attachment feature configured in use for releasable engagement to a corresponding side portion of the first bandage portion;
permanently attaching the first end of each second bandage portion member at or adjacent a corresponding side portion of the heel portion of the first bandage portion such that a central portion of the heel portion is free from contact or overlap with the second bandage portion members.

14. A method, comprising:
providing the single piece foot compression garment as claimed in claim 1;
positioning the first bandage portion such that the sole portion is positioned adjacent a sole of a foot of a user, and the heel portion is positioned adjacent a heel of a foot of a user;
extending the pair of opposed side portions across an upper portion of the foot of the user to provide a predetermined compressive force to an underlying region of the foot, and engaging the at least one first attachment feature of the second end of the first side portion to the second side portion; and
extending each of the second bandage portion members across the dorsum and anterior ankle of the foot of a user to provide a predetermined compressive force to the underlying region of the foot, and securing the attachment feature of the at least one second bandage portion member to a corresponding side portion of the first bandage portion.
